(19) United States
(12) Patent Application Publication
(10) Pub. No.: US 2025/0065016 A1
(43) Pub. Date: Feb. 27, 2025

(54) METHOD AND KIT FOR TREATING A DAMAGED CONNECTIVE TISSUE

(71) Applicant: Reddress Ltd., Pardes Hana (IL)

(72) Inventor: Alon KUSHNIR, Givat Ada (IL)

(21) Appl. No.: 18/723,323

(22) PCT Filed: Dec. 20, 2022
(86) PCT No.: PCT/IL2022/051350

§ 371 Date: Jun. 21, 2024

(30) Foreign Application Priority Data
Dec. 22, 2021 (IL) 289281

(51) Int. Cl. A61L 27/36; A61L 27/54

(52) U.S. Cl. CPC A61L 27/3616; A61L 27/54; A61L 2300/418; A61L 2300/42; A61L 2430/10

(57) ABSTRACT

The present disclosure provides a method and a kit for inducing growth or regeneration of a damaged connective tissue of a subject, or the treatment thereof by applying a clot of blood that is withdrawn from the subject. The connective tissue can be tendon. By one implementation of the present invention, the blood that is withdrawn from the subject is introduced to a lumen of a connective tissue enveloping hollowed element that envelopes the damaged portion of the connective tissue while controllably coagulating it within the lumen to form a clot on the damaged portion of the connective tissue. e.g. by mixing it with coagulation initiators shortly prior to the introduction of the blood into the lumen. The clot, while in physical contact with the damaged portion of the connective tissue, enhances the rehabilitation of the connective tissue and may partially or fully restore its functionality.

### TECHNOLOGICAL FIELD

The present disclosure is in the field of medical treatment of damaged connective tissues.

### BACKGROUND ART

References considered to be relevant as background to the presently disclosed subject matter are listed below:
WO 2019/058373
WO 2019/058375
U.S. Pat. No. 9,180,142

Acknowledgement of the above references herein is not to be inferred as meaning that these are in any way relevant to the patentability of the presently disclosed subject matter.

### GENERAL DESCRIPTION

The present disclosure provides a method and a kit for inducing growth or regeneration of a damaged connective tissue of a subject, or the treatment thereof by applying a clot of blood that is withdrawn from the subject. The connective tissue can be tendon, for example. By one implementation of the present invention, the blood that is withdrawn from the subject is introduced to a lumen of a connective tissue enveloping hollowed element that envelopes the damaged portion of the connective tissue while controllably coagulating within the lumen to form a clot on the damaged portion of the connective tissue. The clot, while in physical contact with the damaged portion of the connective tissue, enhances the rehabilitation of the connective tissue and may partially or fully restore its functionality.

Thus, an aspect of the present disclosure provides a method for regenerating a connective tissue in or across a damaged connective tissue portion. The method includes fitting a connective tissue enveloping hollowed element having a lumen, such as a connective tissue guidance conduit or to envelope the connective tissue portion such that it resides within said lumen. The connective tissue enveloping hollowed element is designed to envelope the damaged connective tissue portion and to maintain a space between a peripheral wall thereof and the damaged connective tissue to allow introduction of coagulating blood withdrawn from the subject to the space to form a blood clot on the damaged connective tissue. Thus, the method further includes introducing a mixture of whole blood withdrawn from the subject with one or more coagulating agents and/or anti-anti coagulating agents into said lumen prior to its complete coagulation of the mixture.

It is to be noted, for any aspect of the present disclosure, that the whole blood may be withdrawn from the subject and initially mixed with anti-coagulating agents to prevent its natural coagulation. At a desired timing, the whole blood is mixed with one or more coagulating agents and/or anti-anti coagulating agents to controllably coagulate the whole blood on the damaged connective tissue portion to form the desired blood clot thereon.

In some embodiments of the method, said fitting comprises placing a film or sheet over said portion and enveloping said portion with said sheet to form said connective tissue enveloping hollowed element.

Yet another aspect of the present disclosure provides a method for inducing growth of connective tissue fibers across a damaged connective tissue portion. The method includes fitting a connective tissue enveloping hollowed element having a lumen, such as a connective tissue guidance conduit to envelope said connective tissue portion such that it resides within said lumen; and introducing a mixture of whole blood withdrawn from the subject with one or more coagulating agents and/or anti-anti coagulating agents into said lumen prior to its complete coagulation of the mixture.

In some embodiments of the method, the connective tissue enveloping hollowed element is a connective tissue guidance conduit.

In some embodiments of the method, the damaged connective tissue portion comprises cut connective tissue fibers.

In some embodiments of the method, the connective tissue portion includes two cut connective tissue stumps that are surgically joined or brought into proximity.

In some embodiments of the method, said fitting includes placing a film or sheet over said portion and enveloping said portion with said sheet to form said conduit.

In some embodiments of the method, said fitting comprises introducing the two connective tissue stumps into said connective tissue enveloping hollowed element, one from each end of the conduit; and bringing the two stumps into proximity to one another within said connective tissue enveloping hollowed element.

In some embodiments of the method, said bringing comprises securing the two stumps within said connective tissue enveloping hollowed element by suturing.

In some embodiments of the method, said introducing comprises filling a portion of the conduit's lumen with the mixture such that it comes into contacts with said connective tissue portion.

In some embodiments of the method, the connective tissue portion includes two cut connective tissue stumps and said introducing is carried out such that said mixture comes into tight contact with said stumps.

In some embodiments, the method further includes saturating the blood, prior to said introducing, with an oxygen-rich gas, e.g., oxygen-enriched air or pure oxygen.

In some embodiments, the method further includes mixing the blood, prior to said introducing, with one or more autologous cells. In some embodiments, said one or more autologous cells comprise Schwann cell.

In some embodiments of the method, the connective tissue is a tendon or a ligament. For example, the connective tissue can be the anterior cruciate ligament, or any tendon associated in the knee.

Yet another aspect of the present disclosure provides a method for treating damaged connective tissue of a subject or facilitating connective tissue regeneration, regrowth or rehabilitation process having a cut connective tissue with first and second connective tissue stumps. The method includes fitting and securing, e.g. by suturing, over said first connective tissue stump a first open end portion of a connective tissue guidance conduit, and fitting and securing over said second connective tissue stump a second open end portion of said connective tissue guidance conduit. The connective tissue guidance conduit defines a lumen extending between said first and second open end portions. The method further includes mixing whole blood withdrawn from the subject with one or more coagulating agents and/or anti-anti coagulating agents and introducing it into said lumen prior to its complete coagulation, namely while it is still in a flowable form, and permitting the blood to coagulate within said lumen.

In some embodiments of the method, said securing comprises suturing said first and second connective tissue stumps to the first and second open end portions, respectively.

In some embodiments of the method, said introducing comprises filling a portion of the lumen with blood such that it contacts both first and second connective tissue stumps, namely filling the gap or space between the first and second connective tissue stumps with blood and creating a continuous matrix of connective tissue stumps and blood.

In some embodiments, the method further includes saturating the blood, prior to said introducing, with oxygen.

In some embodiments, the method further includes, prior to said introducing, adding to or saturating the blood with keratin, stem cells (of all types), growth factors, Collagen, bone marrow or other gases or materials.

In some embodiments of the method, said introducing comprises inserting a blood applicator into the lumen and injecting the blood into the lumen by said blood applicator, e.g. a syringe.

In some embodiments, the method further includes mixing the blood, prior to said introducing, with one or more autologous cells. In some embodiments, said one or more autologous cells comprise Schwann cell.

In some embodiments of the method, the connective tissue guidance conduit is made of or of the type of collagen, polyglycolic acid (PGA), polycaprolactone (PCL), polyvinyl alcohol (PVA), and porcine small intestine submucosa (SIS).

In some embodiments of the method, the connective tissue is a tendon or a ligament.

Yet another aspect of the present disclosure provides a kit for treating, inducing growth, or regenerating a damaged connective tissue portion of a subject. The kit comprising a connective tissue enveloping hollowed element having a lumen for enveloping said damaged connective tissue portion such that said portion resides in at least a portion of said lumen: one or more blood withdrawal devices for allowing withdrawal of blood from the subject: one or more blood collection receptacles for receiving the blood withdrawn from the subject: a coagulation assembly configured for permitting mixture of the withdrawn blood with one or more coagulation agents for initiating coagulation process of the withdrawn blood; and an applicator for introducing the incomplete coagulated blood into said lumen.

In some embodiments of the kit, the one or more collection receptacles are vacuum tubes.

In some embodiments of the kit, the coagulation assembly comprises a volume for introducing the withdrawn blood, said volume is contactable with one or more coagulation agents or anti-anticoagulation agents.

In some embodiments, the kit further includes one or more anticoagulation agents for mixing with the withdrawn blood.

In some embodiments of the kit, the one or more anti-coagulation agents are comprised within said one or more blood collection receptacles.

In some embodiments of the kit, the anticoagulation agent is selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.

In some embodiments of the kit, the one or more coagulation agents or anti-anticoagulating agents are selected from: Kaolin, Ca²⁺, e.g. in the form of calcium salts such as Calcium Gluconate, Mg²⁺, negatively charged phospholipid (PL) and protamine sulfate.

In some embodiments, the kit further includes a film or sheet for placing over said damaged connective tissue portion for forming said connective tissue enveloping hollowed element.

In some embodiments of the kit, the connective tissue enveloping hollowed element is a connective tissue guidance conduit.

In some embodiments of the kit, said damaged connective tissue portion is a cut connective tissue having a first and second connective tissue stumps.

In some embodiments, the kit further includes securing or retaining elements for securing said damaged connective tissue portion to the connective tissue enveloping hollowed element.

In some embodiments of the kit, the connective tissue is a tendon or a ligament.

In some embodiments, the kit is intended for use in a method for treating, inducing growth, or regenerating a damaged connective tissue portion. The method includes (i) enveloping said damaged connective tissue portion with a connective tissue enveloping hollowed element having a lumen such that said damaged connective tissue portion resides in said lumen; (ii) withdrawing whole blood from the subject: (iii) mixing the subject's blood with one or more coagulation agents or anti-anticoagulation agents: (iv) prior to complete coagulation of the blood, introducing the blood with the coagulation agent into said lumen; and (v) permitting the blood to coagulate in said lumen.

In some embodiments, the kit is intended for use in a method according to any one of the above-described embodiments of the method.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1D are schematic illustrations exemplifying different stages of the method of inducing growth or regenerating a damaged or cut connective tissue according to aspects of the present disclosure.

FIGS. 2A-2C are schematic illustrations exemplifying different stages of the method of inducing growth or regenerating a damaged connective tissue according to an aspect of the present disclosure.

### DETAILED DESCRIPTION

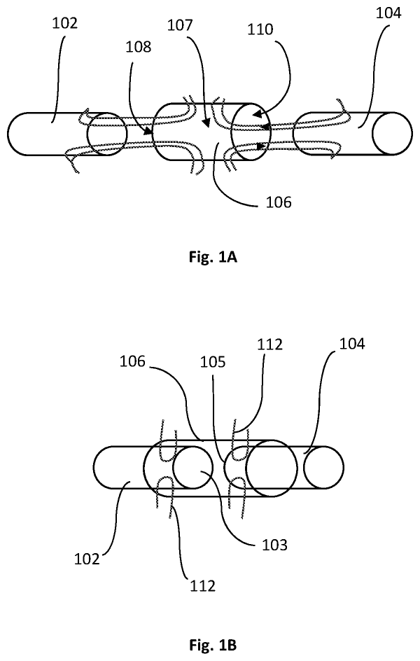

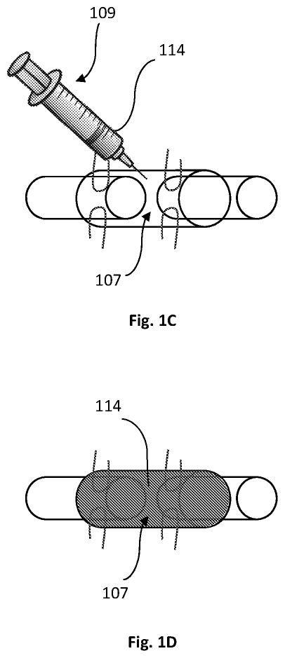

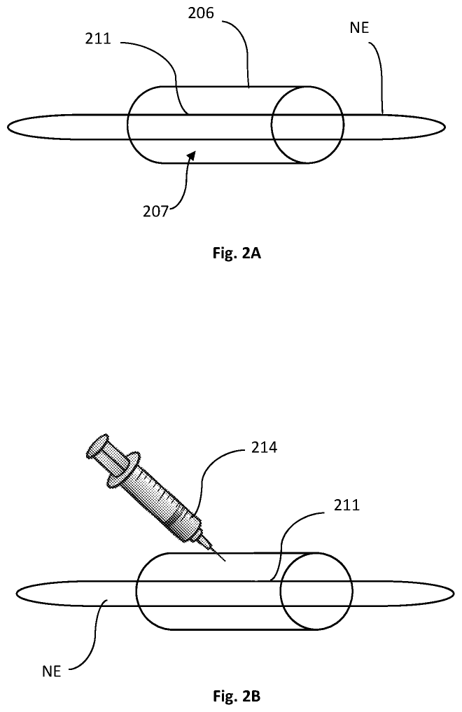

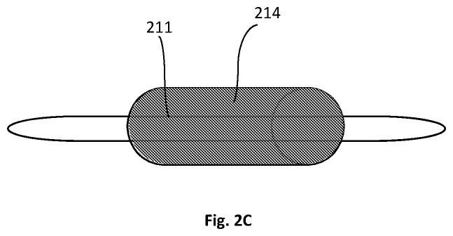

Reference is being made to FIGS. 1A-1D, which are schematic illustrations exemplifying different stages of the method of inducing growth or regenerating a damaged or cut connective tissue according to aspects of the present disclosure. FIG. 1A shows a first and second stumps 102 and 104 of a cut connective tissue of a subject being treated. A connective tissue guidance conduit 106 is a hollowed element having a lumen 107 extending between first and second open ends 108 and 110 and is configured to receive each of the stumps 102/104 through a respective open end such that the connective tissue guidance conduit 106 is fitted over the first and second stumps 102 and 104. In FIGS. 1A-1D and FIGS. 2A-2B, the connective tissue guidance conduit is presented as transparent for ease of description, however it is to be noted that the connective tissue guidance conduit can be made of materials that have any degree of transparency between full transparency and full opaque. In FIG. 1A the connective tissue stumps 102/104 are being introduced via the first and the second open ends 108 and 110, respectively, and being secured, e.g. by suturing process such that portions of the connective tissue guidance conduit envelopes the stumps. FIG. 1B shows the stumps 102 and 104 within the connective tissue guidance conduit 106 and secured in position by securing elements 112 such that a portion that includes the end of each of the stumps 103 and 105 is within the lumen of the connective tissue guidance conduit 106 and each of the stumps extends beyond the respective opening. A mixture 114 of whole blood withdrawn from the subject with one or more coagulating agents and/or anti-anti coagulating agents is introduced into the lumen 107, prior to the complete coagulation of the mixture, namely when the blood is still in a flowable form, as can be seen in FIG. 1C. The introduction of the mixture into the lumen can be performed by a syringe 109 or any suitable means. The lumen 107 is filled with the mixture such that the mixture contacts at least portions of each of the stumps. FIG. 1D shows that the mixture forms a continuous matrix linking the two stumps 102/104. The coagulation process of the mixture proceeds when it is within the lumen 107 until a clot is formed that contacts or envelopes one or two of the stumps 102/104. To retain the mixture within the lumen 107, the first and the second open ends 108 and 110 may be sealed, e.g. by a cloth, a bandage or any suitable sealing means.

Reference is now being made to FIGS. 2A-2C, which are schematic illustrations of a non-limiting example of an embodiment according to an aspect of the present disclosure. FIG. 2A shows a connective tissue guidance conduit 206 that is fitted over a damaged portion 211 of a connective tissue NE of a subject. Once the connective tissue guidance conduit 206 is in place, a mixture 214 of whole blood withdrawn from the subject with one or more coagulating agents and/or anti-anti coagulating agents is introduced into a lumen 207 of the connective tissue guidance conduit 206, prior to the complete coagulation of the mixture, namely when the blood is still in a flowable form, as can be seen in FIG. 2B. The mixture 214 fills the lumen 207 such that it contacts or envelopes the damaged portion 211 of the connective tissue NE and is maintained in the lumen to form a clot that contacts or envelopes the damaged portion 211 of the connective tissue NE, as can be seen in FIG. 2C. In time, the clot induces regeneration or growth of the damaged connective tissue.

1-21. (canceled)

22. A method for treating, inducing growth, or regenerating a connective tissue in or across a damaged connective tissue portion, comprising:
fitting a connective tissue guidance conduit having a lumen over said connective tissue portion such that said portion is within said lumen;
introducing a mixture of (i) whole blood withdrawn from the subject and (ii) one or more coagulating agents and/or anti-anti coagulating agents into said lumen prior to the complete coagulation of the mixture.

23. The method of claim 22, wherein said fitting comprises placing a film or sheet over said portion and enveloping said portion with said sheet to form said conduit.

24. The method of claim 22, wherein the damaged connective tissue portion comprises cut connective tissue fibers.

25. The method of claim 22, wherein said connective tissue portion includes two cut connective tissue stumps that are surgically joined or brought into proximity.

26. A combination for use in regenerating a connective tissue in or across a damaged connective tissue portion, comprising:
a connective tissue guidance conduit configured for enveloping said connective tissue portion;
one or more coagulating agents and/or anti-anti coagulating agents; and
means for introducing a mixture of (i) whole blood withdrawn from the subject and (ii) one or more coagulating agents and/or anti-anti coagulating agents, into a lumen defined by said conduit when enveloping said connective tissue portion prior to the complete coagulation of the mixture.

27. The combination of claim 26, wherein said connective tissue guidance conduit is a film or sheet configured for placement over and enveloping said portion to form said conduit.

28. The combination of claim 26, for use in regenerating cut connective tissue fibers.

29. The combination of claim 26, for use in regenerating two cut connective tissue stumps that are surgically joined or brought into proximity.

30. The combination of claim 26, comprising a blood applicator configured for injecting the blood into said lumen.

31. A kit for use in inducing growth or regeneration of a damaged connective tissue portion of a subject, the kit comprising:
a connective tissue enveloping hollowed element for enveloping a damaged connective tissue portion;
one or more blood withdrawal devices for withdrawing blood from the subject;
one or more blood collection receptacles for receiving the blood withdrawn from the subject;
one or more coagulating agents and/or anti-anti coagulating agents;
a coagulation assembly configured for mixing the withdrawn blood with the one or more coagulation agents and/or anti-anti coagulating agents for initiating a coagulation process of the withdrawn blood; and
an applicator for introducing the incomplete coagulated blood into said lumen.

32. The kit of claim 31, for use in a method that comprises:
fitting said element over said damaged connective tissue portion to envelope said tissue portion to thereby define a lumen, and
introducing a mixture of (i) whole blood withdrawn from the subject and (ii) one or more coagulating agents and/or anti-anti coagulating agents into said lumen prior to complete coagulation of the mixture.

33. The kit of claim 31, wherein said one or more collection receptacles are vacuum tubes.

34. The kit of claim 31, wherein the coagulation assembly comprises a volume for introducing the withdrawn blood, said volume comprises one or more coagulation agents or anti-anticoagulation agents.

35. The kit of claim 31, wherein the coagulation agents or anticoagulating agents are selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin, oxalate, Kaolin, Ca²⁺, Mg²⁺, negatively charged phospholipid (PL) and protamine sulfate.

36. The kit of claim 31, wherein said element is a film or sheet for placing over and enveloping said damaged connective tissue.

37. The kit of claim 31, wherein said connective tissue enveloping hollowed element is a tissue guidance conduit.

38. The kit of claim 31, comprising securing elements for securing said damaged connective tissue portion to the connective tissue enveloping hollowed element.

39. The kit of claim 31, for use in a method for treating, inducing growth, or regenerating a damaged connective tissue portion, which comprises:
(i) enveloping said damaged connective tissue portion with a connective tissue enveloping hollowed element having a lumen such that said damaged connective tissue portion resides in said lumen;
(ii) withdrawing whole blood from the subject;
(iii) mixing the subject's blood with one or more coagulation agents or anti-anticoagulation agents;
(iv) prior to complete coagulation of the blood, introducing the blood with the coagulation agent into said lumen; and
(v) permitting the blood to coagulate in said lumen.
